(19) United States
(12) Patent Application Publication
(10) Pub. No.: US 2023/0414316 A1
(43) Pub. Date: Dec. 28, 2023

(54) OPHTHALMOLOGICAL IMPLANT WITH DIGITAL PRODUCT IDENTIFIER, AND METHOD FOR PRODUCING THE SAME

(71) Applicant: CARL ZEISS MEDITEC AG, Jena (DE)

(72) Inventors: Mario Gerlach, Glienicke-Nordbahn (DE); Benjamin Schreiber, Berlin (DE); Jennifer-Magdalena Masch, Ratzeburg (DE); Thorben Badur, Berlin (DE); Andre Wolfstein, Berlin (DE)

(21) Appl. No.: 18/252,249

(22) PCT Filed: Nov. 10, 2021
(86) PCT No.: PCT/EP2021/081210

§ 371 Date: May 9, 2023

(30) Foreign Application Priority Data
Nov. 10, 2020 (DE) 10 2020 214 126.6
Jun. 15, 2021 (DE) 10 2021 206 092.7

(51) Int. Cl. A61B 90/94; A61F 2/16

(52) U.S. Cl. CPC A61B 90/94; A61F 2/16; A61F 2250/0097; A61F 2250/0089

(57) ABSTRACT

The invention relates to an ophthalmological implant (100) with an optically imaging element (110), a digital product identifier (130) being arranged on the optically imaging element. The invention additionally relates to a corresponding method for producing the implant and to a machine reading system (200) for detecting and decoding the digital product identifier. The aim of the invention is to provide an ophthalmological implant and a method for producing same, said method allowing a unique and complete product identifier and a check thereof using simple means at any point in time. This is achieved by an ophthalmological implant with a digital product identifier (130) which is implemented by means of an encoded point grid (135) of identifier points (57), said point grid being machine-readable in the visible light range and having one irregular semi-random character.

### RELATED APPLICATIONS

This application claims priority from Application PCT/EP2021/081210 filed Nov. 10, 2021, and claims priority from DE Patent Application No. 10 2021 206 092.7 filed Jun. 15, 2021 and DE Patent Application No. 10 2020 214 126.6 filed Nov. 10, 2020, each of which are incorporated by reference in their entireties in this application.

### TECHNICAL FIELD

Example embodiment of the present invention relate to an ophthalmological implant comprising an optically imaging element and for example comprising a haptic adjoining the optically imaging element, with a digital product identification being arranged on the optically imaging element. The present invention also relates to a corresponding method for producing same and a machine reading system for recording and decoding the digital product identification.

### BACKGROUND

Ophthalmological implants, for example commercial intraocular lenses (IOL), are usually identified by labels on the primary and secondary packaging. In addition to other manufacturer information, it is possible to find the type of IOL and its refractive power on the label. The correct treatment of the patient therefore assumes that the packaged and supplied lens corresponds in terms of its properties to the specifications on the label. In this context, the user must rely on the manufacturer since an unambiguous identification and a check in the operating theater only on the basis of the visual lens features are difficult. This type of identification harbors the risk of mix-ups if an ophthalmological implant, for example an intraocular lens, is placed in the wrong packaging. Under certain circumstances, this may lead to explanations and product recalls as a consequence.

Intraocular lenses and other ophthalmological implants are medical devices where traceability is a key requirement. In current products, the lens or implant packaging contains a unique device identifier (UDI) in the form of a bar code, a data matrix code, or a radio-readable microchip. Once the ophthalmological implant has been introduced into the eye, it can no longer be identified accordingly.

Methods and devices have therefore been proposed for individually labelling the ophthalmological implants, for example in US 2006/0001828 A1, in which—outside an optically effective zone—ophthalmological lenses such as contact lenses or intraocular lenses are labelled by application of a matrix code, or other laser-engraved matrix codes in the haptic close to the lens or else farther away. However, labelling outside the optically effective zone is difficult to see in the case of ophthalmological implants once these have been implanted. Moreover, marking the haptic using a laser-engraved matrix code requires a corresponding laser system.

This geometric region of the code of ophthalmological implants, for example of intraocular lenses, according to the prior art is not visually accessible in the eye in the implanted state:

The iris, even when medically dilated, blocks the view of the haptic region. Here, it is desirable to render the code accessible, for example in biometrics or via a microscope (e.g., slit lamp or surgical microscope). According to the ISO 11979-2 standard, which describes the current requirements for ophthalmic lenses, markings on the optically imaging element of the IOL are permissible if a clear zone of 4.4 mm is observed. Thus, for example, a toric mark outside the 4.4 mm region is often used to align toric IOLs in the eye, and these marks are visually accessible with some effort.

In contrast, WO 2009/124838 A2 describes an ophthalmological implant with a marking that is also possible on an optically imaging element. However, a fluorescent dye with an emission maximum outside the light spectrum visible to humans or an absorbing dye with an absorption maximum outside the light spectrum visible to humans is used for marking in this case. However, the method used here is technically very complex and requires an additional biocompatible fluorescent dye or absorbing dye and a complex fluorescence excitation or detection system.

Both methods described here also need to be incorporated in the manufacturing chain in such a way that there is no possibility of incorrect labelling of the IOLs as a result of operating or programming errors. Both require additional manufacturing steps using additional tools to implement the markings.

### SUMMARY OF THE INVENTION

Example embodiments of the invention include an ophthalmological implant, for example an intraocular lens, and a method for producing same, which allow a clear and complete product identification (UDI), for example type, refractive power, serial number, batch number on the intraocular lens, and verification of same using simple instruments and at any time.

An ophthalmological implant, for example an intraocular lens, comprises an optically imaging element, in particular a central optical lens, with an optically effective zone and for example with a haptic adjoining the optically imaging element. Ophthalmological implants usually comprise a haptic for the appropriate fixation of these implants in a patient's eye. In special cases, however, an ophthalmological implant is complete and implantable even without a haptic.

On the optically imaging element of the ophthalmological implant, for example within the optically effective zone, there is a digital product identification of the ophthalmological implant, in particular for example the type and the refractive power and/or a database key (i.e., a unique identifier).

If the digital product identification comprises a database key in the sense of a unique identifier, then the implant-specific product information has been assigned in advance in a database system to this database key. By reading out the database key on the ophthalmological implant (e.g., using the machine reading system described below, which in that case is for example connected directly to the database system), a database query of the implant-specific product information can be carried out. Using this procedure, comprehensive and clear product information can be made available with short coding lengths.

If a database key is arranged on the ophthalmological implant as a digital product identification for unique identification purposes, then usually only this database key is “stored” on the ophthalmological implant—since the database key ultimately provides access to unique information, stored or storable in great detail in the database system itself, about the specific ophthalmological implant.

The database system is for example made available in a data network or a data cloud. For example, the manufacturer stores the implant-specific product information for each database key, that is to say for each unique identifier, in this database system.

However, it is also conceivable that, in addition to this database key, the most important product information, such as type and refractive power, is arranged as digital product identification regardless, with the result that a treating physician, for example, can get hold of these most important product identifications even without a connection to the database system.

According to the invention, this digital product identification is implemented by use of a “coded marker” which is machine-readable in the visible light range and is realized here in the form of a coded point grid with pseudo-random irregular character made of marking points. In this case, “pseudo-random irregular” means that there are a number of different defined deviations of the marking points from a fixed reference point, which would produce a regular character of the point grid. As a result, it is possible to minimize grating diffraction effects which would exist in the case of a regular point grid and would lead to an impairment of imaging by use of such an ophthalmological implant, and hence, for example, to an impairment of vision with a corresponding intraocular lens.

In this case, the optically effective zone of the optically imaging element, in which the digital product identification is for example arranged, is a zone determined by the pupil opening under normal lighting conditions.

To begin with, a distinction should be made between two regions for the marking of the optically imaging element, in particular those of the optically effective zone:

The central optical zone is the central region with a diameter of 4.4 mm; marking in this region is currently not ISO-compliant.

The peripheral optical zone is the outer region of the optically imaging element, in the case of which the central optical zone with a diameter of 4.4 mm remains free; marking in this outer region is currently ISO-compliant. The optically effective zone of the optically imaging element is thus part of the central optical zone, in which marking has not been carried out/has not been allowed to date: This is because, to date, the use of markings according to the prior art has not rendered this possible without significantly impairing visual quality for the patient wearing the implant (the IOL).

Here, marking points of the machine-readable coded point grid are not points in the mathematical sense, but have a size: As a rule, they are round and characterized by a diameter. In an embodiment, however, they can assume an elliptical shape—that is to say they have a length that is significantly different from the width when viewed from above. Other geometric shapes, such as squares and rectangles, are also possible.

Thus, optical codes, for example data matrix codes, on or in the optically imaging element of an ophthalmological lens are described in this invention. The systems and methods outlined herein are based on an IOL equipped with a coded marker. However, the invention does not exclude other ophthalmological implants that need to be positioned in the eye, for example capsular tension rings, stents and ICLs.

Equipping the implant with a coded marker, also known as a “tag”, as an identifier offers various advantages. It supports the surgical workflow. However, it also allows traceability of the implant, from production, through logistics, implantation, in-vivo and, optionally, post-explanation for complaint management.

The focus here is therefore on, inter alia, the link to production and surgical equipment and to diagnostics. In order for this to be possible, the coded marker is made visually accessible even in the implanted state.

In an example embodiment of the ophthalmological implant according to the invention, the machine-readable coded point grid made of marking points is arranged centrally within the optically effective zone of the optically imaging element. As a result, it is visually accessible at all times, even in the implanted state, and rendered visible and readable by using an appropriate machine reading system.

Even if, for the coded point grid made of marking points, a central location within the optically effective zone of the optically imaging element is particularly advantageous in terms of accessibility, the coded point grid made of marking points can be located at any position within the optically effective zone or else outside of the optically effective zone.

The object is therefore achieved to the effect of the ophthalmological implant being associated with a digital identifier in the form of a machine-readable coded point grid that can be read in the visible light range, but which, contrary to the usual interpretation of the term “point grid”, precisely does not have any regularities in a form that could lead to grating diffraction effects. It is for this reason that this point grid is even arrangeable centrally within the optically effective zone of the IOL: By way of its structure, it is designed in such a way that the patient does not perceive any negative optical effect.

In a specific and particularly advantageous embodiment of the ophthalmological implant according to the invention, the coded point grid is constructed from marking points such that a virtual polar or Cartesian base lattice is arranged on the optically imaging element, for example on the optical zone of the optically imaging element, in such a way that this describes similar sectors or similar cells, each with a defined base lattice point of the sector or the cell, as a result. Such a (virtual) base lattice point of the virtual base lattice can be understood to be the center of the sector or cell.

By contrast, a real marking point of the point grid is arranged in each sector or each cell at a position which has an offset to this base lattice point, the offset in each sector or each cell being in one of four possible directions, which for example run pairwise opposite to each other, and having a defined distance to the base lattice point. Naturally, more than 4 directions of the offset are also possible, depending on the options for the creation of the coded point grid made of marking points and the resolution of a machine reading system intended to read out this coded point grid again. Therefore, this particularly advantageous example embodiment is intended to be read as “at least four possible directions of the offset of the marking point to the base lattice point”.

In an embodiment of the ophthalmological implant according to the invention as just described, a sector or cell with a base lattice point provides four states which are characterized by the respective location of the marking point in one of four positions around the base lattice point. The marking point of the sector or cell can thus in each case assume one of four possible positions around a base point of a sector or cell, as a result of which four different states can be described using this sector or cell.

In a development of this embodiment of the ophthalmological implant, a sector or cell with a base lattice point provides a further state, a fifth state, defined by the absence of a marking point at one of the four possible positions around a base lattice point. This results in five possible states for a sector or cell. This is advantageous in that the coding of a unique identification number into the five possible states of the sectors or cells leads, in the statistical average, to the absence of a marking point in this sector or cell in ⅕ of cases. Thus, the number of actually required marking points is reduced by−⅕. This further reduces potential optical effects of the coded point grid made of marking points.

In a further embodiment of the ophthalmological implant according to the invention, further states are defined in a sector or cell with a base lattice point by way of further possible offset directions and/or further possible defined distances of the offset of the marking point to the base lattice point.

The inventive concept therefore also comprises the additional arrangement of the marking points at further positions within a sector or cell on the base crossing points or further crossing points between the base lattice and offset lattice. By way of example, a sector or cell with a (virtual) base lattice point can generate up to nine geometric states if the base crossing points are also used. How many positions are clearly and reliably identifiable depends on the performance of the method for generating the machine-readable coded point grid made of marking points and on the performance of the machine reading system.

An ophthalmological implant according to the invention is advantageous in which the proportion of the area of the marking points to the total area of the optically imaging element is for example less than 2%, in another example less than 1%, and in a further example less than 0.5%, and/or wherein a proportion of the area of the marking points to the area of the optically effective zone of the optically imaging element is for example less than 8%, in another example less than 4%, and in a further example less than 2%. In this case, the total area of the optically imaging element is typically an area with a diameter of 6 mm, and the area of the optically effective zone of the optically imaging element is a zone determined by the pupil opening under normal lighting conditions, that is to say usually an area with a diameter of approximately 3 mm.

In addition to the option of using the absence of a marking point in a sector or cell to describe the state and thus “economizing” marking points, it is generally expedient to keep the proportion of the area of all marking points in the total area of the optically imaging element and in particular in the area of the optically effective zone of the optically imaging element as small as possible.

By way of its structure, the coded point grid made of marking points, according to the invention, is thus designed in such a way that the patient does not perceive any negative optical effect. To ensure this, the following conditions must be met:
a) It has a pseudo-random, that is to say irregular character, in order to minimize grating diffraction effects (for example, realized by the offset arrangement of marking points to a polar or Cartesian basic lattice),
b) the individual points should be as small as possible, and
c) it should contain as few grid points as absolutely necessary for the content.

In a further example embodiment of the ophthalmological implant according to the invention, the machine-readable coded point grid has structural marking points. In this case, structural marking points are marking points that are characterized by a topology, that is to say are physically raised or physically impressed, with the latter representing the more probable embodiment of these marking points.

These are advantageous for two reasons: Firstly, these points still ensure a certain transparency, that is to say they are not completely opaque absorbers. Secondly, the generation of such structural marking points is able to be integrated relatively easily into the manufacturing process of an ophthalmological implant.

Nevertheless, it is also possible to generate marking points by applying or introducing dyes in order to realize the ophthalmological implant according to the invention.

The contrast of, for example, laser-engraved marking points is usually given by scattering. With relatively recent technologies, a local optical change can be used to also achieve contrasts in addition to scattering and to generate the presented codes:
Nanostructures that act as light traps can be used.
Periodic nanostructures can be used for the purpose of reflection (of selective wavelengths).
Local refractive index changes can be induced, such as those generated in light-adjustable IOLs for example.
Organic and inorganic dyes and absorbers can be used as a coating or in locally fixed fashion, for a wavelength-selective readout of the code. Disperse red 1, which is fixed to the surface by plasma activation, can be used as organic dye in the visible range. Should near-IR readout be intended, Epolight™ 1117 or Epolight™ 1178 (both from Epolin) can be applied locally as a lens coating. An example of inorganic dyes is the use of metal or silicon nanoparticles.
Polychromatic combinations of organic and/or inorganic dyes and absorbers can be used to increase data density, thereby allowing the code size to be reduced.
Photoactivatable dyes which can be anchored in the polymer matrix by local laser-induced light activation are also possible. The lens is soaked in a solution containing the dye (e.g., an organic monomer such as acryloxy fluorescein) and an initiator (if necessary), resulting in diffusion into the material, followed by spatial photofixation and rinsing of the lens to remove unreacted dye.
Non-abrasive methods such as printing or photobleaching are further alternatives to create the desired markers. Micro-inkjet systems or micro-structuring systems can likewise be used. For example, the current Zeiss LUCIA heparin coating process applied to the finished intraocular lens can be modified and augmented with a step in which a dye pattern is covalently bonded to the Polymin linker on the lens surface.

Such alternative approaches are advantageous as they reduce the amount of light which is scattered by the marking points and reaches the retina, causing side effects such as “star bursts” or other dysphotopsia. In the case of wavelength-selective reflection, absorption or refractive index changes, additional functions (chromatic filters, light polarizers and filters, lamps) may be required in the optical readout equipment. However, this is accompanied by a reduction in side effects on the visual impression of the patient. In addition, the markings are then not visible from the outside (which is cosmetically beneficial for the patient). This in turn allows the coded markers to be easily accommodated in the central optic and allows for easier optical access.

Moreover, it is very advantageous for example if the ophthalmological implant according to the invention contains a supplemented product identification which, in addition to the original product identification, has information for checksums and error correction methods.

On account of the huge number of representable states for clear product identification, there is sufficient representation reserve for checksums and error correction mechanisms, which represent additional safety when using such an ophthalmological implant.

The information-theoretical design of the coding schemes for example allows the coding of very long integers in order to enable a unique product identification of many (and different) implants over a long period of time. Due to the possible storage capacity, the coding itself can be protected against incorrect reading using known methods from information technology. These include, for example, checksum or error correction methods.

To improve the recognizability and referencing by a machine reading system, the ophthalmological implant has, in a further embodiment, one or more reference marks at a defined distance from the machine-readable coded point grid made of marking points. These one or more reference marks are therefore arranged in the vicinity of the coded point grid made of marking points and in a defined distance relationship to the latter and form the “control points” for a machine reading system, in relation to which the said machine reading system orientates itself and thereafter is able to very easily correctly localize and read the coded point grid made of marking points.

In different embodiments of the ophthalmological implant, its machine-readable coded point grid made of marking points, or more generally the coded marker, can be arranged on the optically imaging element (i.e., applied to its surface or introduced into its surface) or in the optically imaging element (i.e., in the volume of the optically imaging element) in the process. A combination of both embodiments is also possible.

An example embodiment of the ophthalmological implant according to the invention has an alignment aid. The alignment aid is likewise for example arranged within the optically effective zone. It is furthermore advantageous for example for the alignment aid to contain or consist of the machine-readable coded point grid made of marking points.

A further example embodiment of the ophthalmological implant according to the invention comprises a toric marker which is readable in the view from above and/or a toric marker which is readable in an axial view. The toric marker can likewise be arranged on the surface of the optically imaging element and/or in the volume thereof. Moreover, the toric marker may also contain or consist of the machine-readable coded point grid made of marking points.

A toric marker which is readable in the view from above allows access via camera in production and logistics and via surgical microscope, a slit lamp or biometric equipment in the implanted state. A toric marker which is readable in an axial view (that is to say in a side view) is accessible to tomographic measurements, for example. For example, it can be used with OCT (optical coherence tomography), CT (computed tomography) or MRI (magnetic resonance imaging).

Data matrix codes in the form of a toric marker serve to assist with in vivo lens alignment in the eye during surgery and with rotation traceability. To correct regular astigmatism, toric intraocular lenses have an optical cylinder arranged along a specific axis, the toric axis. The toric axis is indicated by the toric markers. Since the lens alignment in the eye during surgery is implemented with the surgical microscope using software such as Callisto, the necessary lens information can be automatically supplied in vivo. Furthermore, this function is intended not only for toric IOLs but also for monofocal IOLs, allowing for operative in-vivo and post-operative tracking of lens centering and rotation. Here, the data matrix code serves as an optical reference for tracking the lens alignment in vivo.

Thus, various embodiments of coded markers that differ from a standard code are presented here. In particular, the toric marker is visually accessible both in standard routines (biometric equipment, surgical microscopes, slit lamps) and/or in more advanced optical designs (pinhole concepts, central optic designs).

The embodiments with alignment aids and/or toric markers for all ophthalmological implants, in particular for intraocular lenses (toric and non-toric; modular lens alignment), also make it possible to detect postoperative changes in the implant position (inclination, decentering, rotation of all lenses).

Based on tests on an intraocular lens implanted in a human eye model, a marking point size (spot size) of ˜25 μm is preferred for example for the machine-readable coded point grid made of marking points, or more generally for the coded marker. It represents the best compromise between size and contrast visible in the surgical microscope. A conventional axial resolution of ˜5 μm is possible for OCT A. This would be a lower bound for the marking point size; a factor of 2 could be applied via resolution criteria.

Enabling traceability throughout the lifetime of the implant is advantageous in the case of a biomaterial code (thus a coded marker on an ophthalmological implant here). A greater distance between the marking points (lattice size>point size) reduces possible diffraction effects. The use of non-periodic data codes, not only at the level of the arrangement of individual marking points, but also with regard to the overall structure of the coded markers, is for example advantageous. Contrast enhancements such as laser-induced light traps or reflective structures or refractive index changes are likewise advantageous for example to reduce the risk of dysphotopsia.

A machine reading system according to the invention serves to capture and decode the digital product identification in the form of a coded point grid made of marking points, or in general the coded marker, on an ophthalmological implant described here.

The machine reading system comprises a camera system for recording structures of the machine-readable coded point grid made of marking points on the ophthalmological implant, and an analysis unit for capturing and evaluating a camera system recorded image of the structures of the machine-readable coded point grid made of marking points and for decoding the digital product identification of the ophthalmological implant, in particular for example the type and refractive power and/or the database key, for the identification thereof from this image.

Depending on the respective lighting conditions, such a machine reading system can have an illumination system for illuminating a digital product identification of an intraocular lens in the form of a machine-readable coded point grid made of marking points.

It is moreover advantageous for example if the machine reading system also comprises a display and/or output apparatus for displaying and/or outputting the decoded identification data of the ophthalmological implant. Otherwise, however, such a display or output can also be adopted by other apparatuses that can be connected to the machine reading system.

Especially if the digital product identification of the ophthalmological implant contains a database key, it is moreover advantageous for example if the machine reading system is connected to a database system. This database system assigns the implant-specific product information to a database key, which is a unique identifier. A database query of implant-specific product information can be carried out by the database key stored as the digital product identification of the ophthalmological implant being read by the machine reading system. Using this procedure, comprehensive and clear product information can be made available with short coding lengths. The database system is for example made available in a data network or a data cloud. For example, the manufacturer stores the implant-specific product information for each unique identifier in this database system.

In specific embodiments, the machine reading system according to the invention is part of a surgical microscope or a slit lamp.

In this case, the unique product identification can be stored and/or processed by the machine reading system, and can be made available digitally for other services. Such a machine reading system can be located in the production of the ophthalmological implant, for example in a production of intraocular lenses for quality monitoring and/or in the practice of the implanting and/or controlling ophthalmologist, for the purpose of checking the ophthalmological implant.

In a method according to the invention for producing an ophthalmological implant with a digital identifier as described above, the machine-readable coded point grid made of marking points is generated for digital product identification purposes on the ophthalmological implant, during or after the production of the latter.

The identifier is therefore connected to the ophthalmological implant, for example to an intraocular lens (IOL), during the manufacturing process and can then be read, stored, and processed by optical instruments during further subsequent steps in the cataract operation and in the implanted state.

In an example embodiment of the method according to the invention for producing an ophthalmological implant, the latter is labelled during the production of the ophthalmological implant
either in an early phase, that is to say before the production of the ophthalmological implant is complete, with the machine-readable coded point grid made of marking points,
or directly after the production has been completed, but still within the same step with basically the same tool used to produce the ophthalmological implant, with the machine-readable coded point grid made of marking points.

An embodiment of the method according to the invention for producing an ophthalmological implant, in which the machine-readable coded point grid made of marking points is introduced into the surface of the ophthalmological implant using a CNC-controlled drilling or milling tool during or after the production of the said implant, with the drilling or milling tool to this end for example having a tool diameter of less than 0.4 mm.

Usually, cutting turning methods, in particular diamond turning methods, are used for the production of ophthalmological implants themselves, that is to say CNC-controlled milling tools are used for this purpose. Ideally, the ophthalmological implant is now digitally labelled without the tool having to be changed for this purpose, since every change of machine and hence change of location of the ophthalmological implant can naturally be a source of mix-ups. However, if the ophthalmological implant can be digitally labelled with basically the same tool also used for the manufacture thereof, then this renders possible a method in which the data actively “collected” during the manufacturing process or used during the manufacturing process of the ophthalmological implant are also coded into this ophthalmological implant during or directly after manufacture. A mix-up of the data set or ophthalmological implant is thus precluded.

In this case, the machining for digital product identification is carried out with small tools (for example with a diameter <0.4 mm) in a surface-near region.

In an alternative embodiment of the method according to the invention for producing an ophthalmological implant, the machine-readable coded point grid made of marking points is applied either by use of laser processing by ablation or disruption or by application of printing methods, for example using biocompatible chromophores or pigments, which are usually situated in a matrix that bonds covalently to the lens material.

In an embodiment of the method according to the invention for producing an ophthalmological implant, a product identification or a supplemented product identification is in the process converted into grid coordinates for the physical product identification using a machine-readable coded point grid made of marking points.

The unique product identification is connected to the ophthalmological implant by use of the point grid made of marking points according to the invention. To this end, the unique product identification is initially supplemented with the information for checksum or error correction methods, if this is intended. The precise procedure here depends on the method selected. Thereafter, the supplemented unique product identification is converted into the grid coordinates for the physical product identification. The identification can now be transferred to the product, into the point grid made of marking points, using the grid coordinates.

Not least, a further embodiment of the method according to the invention provides, during or after the generation of the machine-readable coded point grid made of marking points for digital product identification, for the said machine-readable coded point grid to be stored in a manufacturer database system which can be linked to an electronic patient file and/or another data collection point for medical or official purposes.

The unique product identification is generally generated by the manufacturer himself or, if necessary, by a certification body during the manufacture of the ophthalmological implant.

According to the invention, this identification is then connected to the product and stored in a database system. This database system can be created in self-contained fashion by the manufacturer and/or at a public data collection point for medical or official purposes, or can be transferred from the manufacturer to a corresponding data collection point. This product identification can also be stored in an electronic patient file.

In an important embodiment of the method according to the invention for producing an ophthalmological implant, the product information of the ophthalmological implant is stored in a database system before the machine-readable coded point grid made of marking points for digital product identification is generated and a database key for this product information, which is contained in the digital product identification, is generated.

Such a method step can be implemented instead of the previously cited method step of storing the digital product identification in a database system during or post generation. However, it is also possible for a database key to be created in the database system prior to generation and for the database system to be write accessed during or post generation in order to confirm the actually generated digital product identification, for example the database key, which was generated by the machine-readable coded point grid as marking points on the ophthalmological implant. A comparison for example takes place in the process, and an incorrectly labelled ophthalmological implant is blocked in the event of deviations with respect to the database key.

### BRIEF DESCRIPTION OF THE DRAWINGS

The invention is explained in more detail below by way of example on the basis of the accompanying drawings, which also disclose features essential to the invention. In detail:

FIG. 1 depicts a digital product identification as can be used in an ophthalmological implant according to the invention, here in the first example embodiment of FIGS. 2 and 2a; FIG. 1a depicts an enlarged section of this digital product identification;

FIG. 2 depicts a first example embodiment of an ophthalmological implant according to the invention, here an intraocular lens; FIG. 2a depicts an enlarged image representation of the product identification on the optically imaging element, here on the lens body of an intraocular lens;

FIG. 3 depicts a second example embodiment of an ophthalmological implant according to the invention; FIG. 3a depicts an enlarged image representation of the product identification on the optically imaging element;

FIG. 4 depicts a third example embodiment of an ophthalmological implant according to the invention; FIG. 4a depicts an enlarged image representation of the product identification on the optically imaging element;

FIG. 5 depicts a fourth example embodiment of an ophthalmological implant according to the invention; FIG. 5a depicts an enlarged image representation of the product identification on the optically imaging element;

FIG. 6 depicts a fifth example embodiment of an ophthalmological implant according to the invention; FIG. 6a depicts an enlarged image representation of the machine-readable point grid made of marking points used for digital product identification;

FIG. 7 depicts a sixth exemplary embodiment of an ophthalmological implant according to the invention; FIG. 7a depicts an enlarged image representation of the machine-readable point grid made of marking points used for digital product identification;

FIG. 8 depicts an intensity distribution directly downstream of an area marked with the coded point grid made of marking points of the sixth example embodiment;

FIG. 9 depicts the modulation transfer function for the central point grid made of marking points of the sixth example embodiment;

FIG. 10 depicts a machine reading system according to the invention for capturing and decoding of a coded point grid made of marking points on an ophthalmological implant.

FIG. 11 depicts the use of a coded marker for verification of the implant during implantation and in vivo post implantation;

FIGS. 12a to 12c depict a digital product identification with toric marker and alignment aid according to the prior art and in different example embodiments of the ophthalmological implant according to the invention;

FIGS. 13a to 13c depict digital product identifications with toric marker in the optically effective zone of the optically imaging element in further different example embodiments of the ophthalmological implant according to the invention;

FIGS. 14a to 14c depict a digital product identification with toric marker in further different example embodiments of the ophthalmological implant according to the invention—for use from the view from above and for use in an axial view;

FIGS. 15a to 15c depict how a digital product identification in further different example embodiments of the ophthalmological implant according to the invention;

FIG. 16 depicts different arrangements of the machine-readable coded point grid made of marking points of a digital product identification;

FIGS. 17a and 17b depict a size estimation for M3 and M4 UDI codes, respectively;

FIGS. 18a to 18d depict a manufacturing method of an intraocular lens with a coded marker arranged in the volume of the optically imaging element;

FIGS. 19a to 19c depict an intraocular lens implanted in an ISO eye, having a coded marker on the surface of the optically imaging element and the halo/glare test with this and without this coded marker.

### DETAILED DESCRIPTION

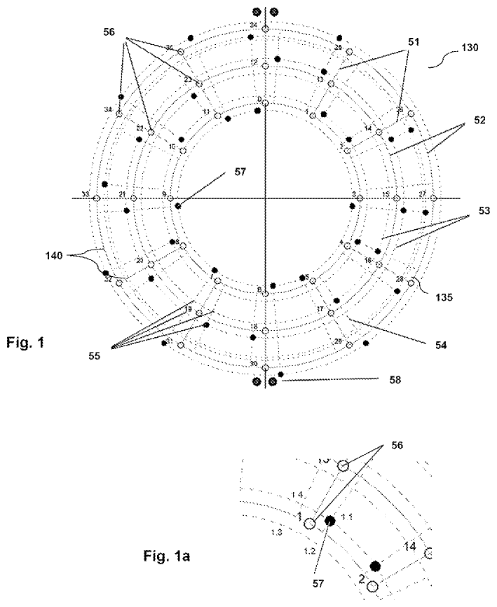

Firstly, FIG. 1 illustrates a digital product identification 130 as can be used in an ophthalmological implant 100 according to the invention, in order to be able to better explain its principles. FIG. 1a shows an enlarged portion of this digital product identification 130. The (machine-readable) coded point grid 135 made of marking points 57 for digital product identification 130, which is shown in FIGS. 1 and 1a, is obtained by the offset arrangement of the marking points 57 to form a polar base lattice, although the explanations also apply in principle to an arrangement of marking points 57 in a Cartesian base lattice (in which a sector 51 is then called a cell 51′).

The polar base lattice shown here consists of three radial zones 52, each with twelve sectors 51. These generate the base lattice points 56, which are purely virtual in nature. The base lattice points are indexed consecutively from 0 to 35 in FIG. 1 and label the respective sector 51 (or the respective cell). Moreover, the grating contains positive and negative offset zones in the sectoral 54 and radial 53 directions. These generate four further crossing points 55 around the base lattice points 56. The marking point 57 can be located on one of these four crossing points 55—the four crossing points therefore represent the possible positions of the marking point 57 in the corresponding sector 51 or in the corresponding cell 51′.

FIG. 1a then shows an enlarged view of a base lattice point 56 and its surroundings or corresponding sector 51. It is apparent here that the four positions 55 are indexed around the base lattice point 56. For the base lattice point 56 with the index 1, these are the positions 1.1, 1.2, 1.3 and 1.4. In this example, the marking point 57 of the sector 51 associated with the base lattice point 56 can assume one of the four different positions 55 1.1, 1.2, 1.3 or 1.4. Thus, in this example, a single base lattice point 56 can assume four states. Hence, 4³⁶=4 722 366 482 869 645 213 696 representable states are obtainable in the case of a total of 36 sectors with four positions 55 each (i.e., four possible states).

In order to improve the recognizability and referencing by a machine reading system 200, a plurality of reference marks 58 are moreover arranged in the vicinity of the point grid 135 made of marking points 57.

In the example of FIG. 1 explained here, the respective marking point 57 can assume one of four possible positions 55 around a base point 56. Thus, a total of 36 marking points 57 are arranged in the polar base lattice.

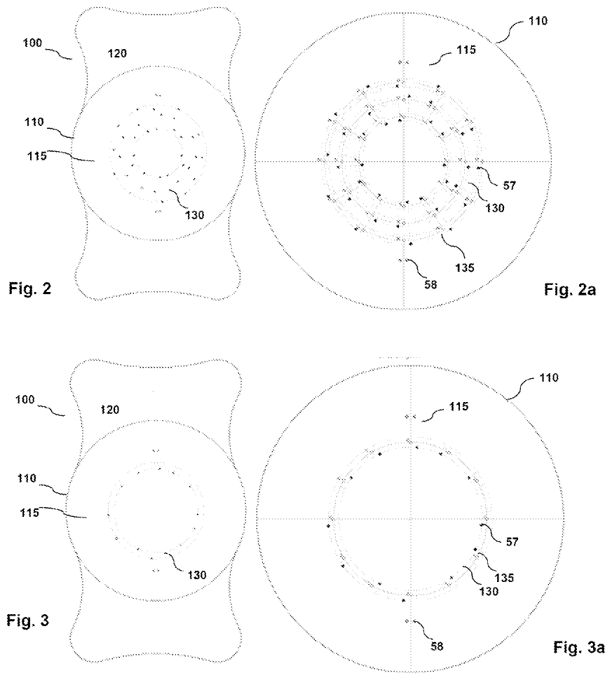

FIG. 2 shows a first example embodiment of an ophthalmological implant 100 according to the invention, here an intraocular lens, with a digital product identification 130; FIG. 2a shows an enlarged image representation of the product identification 130 on the optically imaging element 110, here on the lens body of an intraocular lens. This first example embodiment is a coded point grid 135 made of marking points 57, which uses a polar base lattice and has three zones 52 and twelve sectors 51 per zone 52, each with four states per sector 51. As shown in the example in FIG. 1, this results in 4³⁶ representable states that can be used to store the digital product information. In this example embodiment, the individual point size of a marking point 57 is approximately 0.0025 mm², and hence the proportion of the area of the marking points 57 to the total area of the optically imaging element 110 is 0.3178%.

FIG. 3 shows a second example embodiment of an ophthalmological implant 100 according to the invention; FIG. 3a shows an enlarged image representation of the product identification 130 on the optically imaging element 110 of this ophthalmological implant 100. This second example embodiment is a coded point grid 135 made of marking points 57, which uses a polar base lattice and in this case has one zone 52 with twelve sectors 51, with each sector 51 being able to describe four states. Thus, 4¹²=16 777 216 representable states are obtained.

In this example embodiment, the single point size of a marking point 57 is approximately 0.0025 mm². Hence, the proportion of the area of the marking points 57 to the total area of the optically imaging element 110 is 0.1059%.

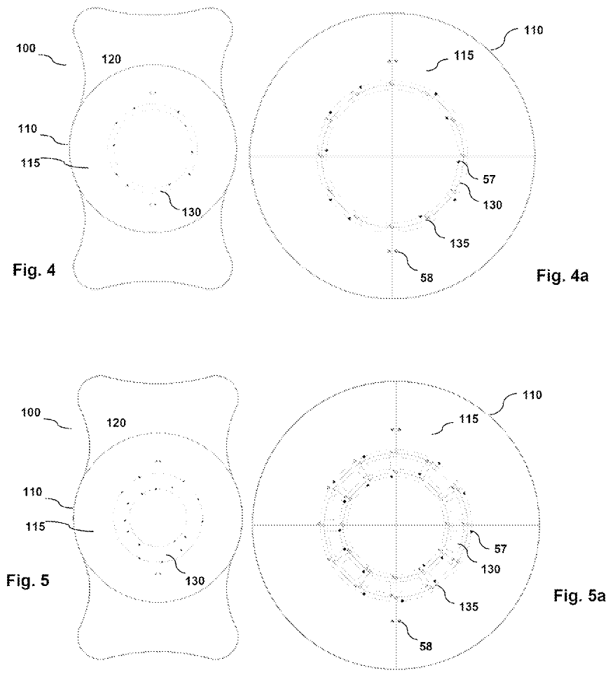

FIG. 4 illustrates a third example embodiment of an ophthalmological implant 100 according to the invention; FIG. 4a illustrates an enlarged image representation of the digital product identification 130 on the optically imaging element 110. This third example embodiment is also a polar base lattice with one zone 52 that contains twelve sectors 51. However, five states can be described by each of these sectors 51 in this case since, in addition to the four possible states that are due to the location of a marking point 57 on one of the four positions 55 that have an offset to the base lattice point 56, the absence of a marking point 57 in the corresponding sector 51 describes a further state. Hence, 5¹²=244 140 625 representable states are obtained.

In this example embodiment, the individual point size of a marking point 57 once again is approximately 0.0025 mm², and hence the proportion of the area of the marking points 57 to the total area of the optically imaging element 110 is 0.1059%. Thus, although the possible storage capacity has increased, the proportion of the area of the marking points 57 to the total area of the optically imaging element 110 has remained the same in comparison with the second example embodiment.

FIG. 5 shows a fourth example embodiment of an ophthalmological implant 100 according to the invention; FIG. 5a once again shows an enlarged image representation of the product identification 130 on the optically imaging element 110 of the ophthalmological implant 100. This fourth example embodiment once again is a polar base lattice, but it has two zones 52 that contain twelve sectors 51 each. Five states can also be described by each of these sectors 51 in this case. Hence, 5¹²=59 604 644 775 390 625 representable states are obtained.

In this example embodiment, the individual point size of a marking point 57 once again is approximately 0.0025 mm², and hence the proportion of the area of the marking points 57 to the total area of the optically imaging element 110 is 0.2119%.

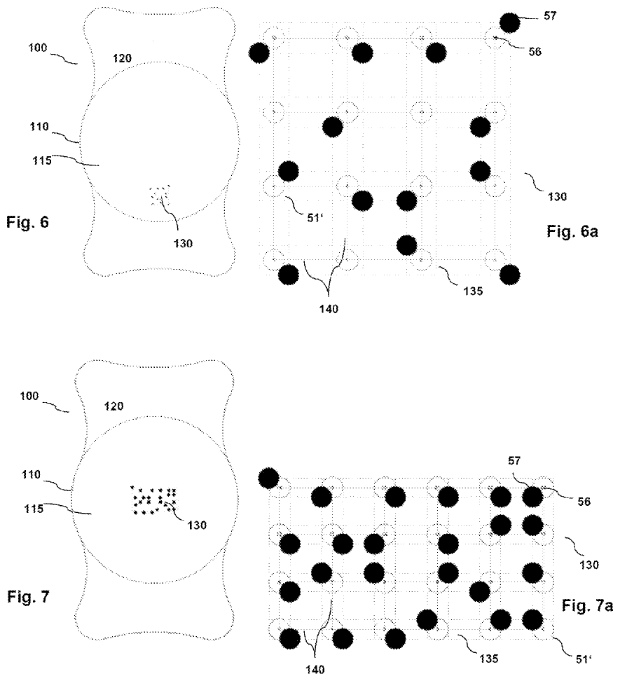

FIG. 6 shows a fifth example embodiment of an ophthalmological implant 100 according to the invention; FIG. 6a shows an enlarged image representation of the machine-readable point grid 135 made of marking points 57 used for digital product identification 130. In this fifth example embodiment, however, use is made of a Cartesian base lattice with an extent of four cells 51′ in a lateral direction x and four cells 51′ in a lateral direction y (i.e., nₓ=4, n_{y}=4), in which five states can be described by each cell 51′, that is to say four states by the location of a marking point 57 on one of the four possible positions 55, which are offset (in each case in a different direction) from the base lattice point 56 of the cell 51′, and an additional state due to the absence of a marking point 57 4¹⁶=4 294 967 296 representable states. In this embodiment, too, the single point size of a marking point 57 is approximately 0.0025 mm². Hence, the proportion of the area of the marking points 57 to the total area of the optically imaging element 110 is 0.1413%.

FIG. 7 illustrates a sixth example embodiment of an ophthalmological implant 100 according to the invention, once again an intraocular lens, and FIG. 7a illustrates an enlarged image representation of the machine-readable point grid 135 made of marking points 57 used for digital product identification 130. In this sixth example embodiment, too, a Cartesian base lattice is used with an extent of six cells 51′ in a lateral direction x and four cells 51′ in a lateral direction y (nx=6, ny=4), in which four states can be described by each cell 51′. Hence, 4 24=281 474 976 710 656 representable states are available for storing the (optionally supplemented) product identification 130. In this example embodiment, the single point size of a marking point 57 is approximately 0.0113 mm². Hence, the proportion of the area of the marking points 57 to the total area of the optically imaging element 110 is 0.96%, and hence significantly higher than in the previous example embodiments.

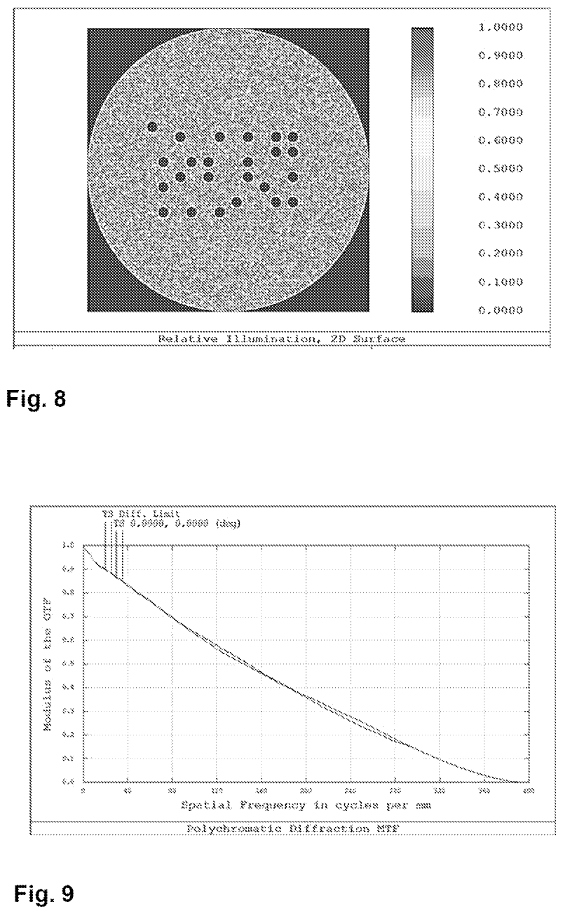

In the sixth example embodiment of FIGS. 7 and 7a (Cartesian base lattice, nₓ=6, n_{y}=4, four states), a Cartesian point grid 135 made of marking points 57 with relatively large marking points 57 in the center of an intraocular lens is illustrated as an optical worst-case example. This pattern was imported as a “user defined obscuration” into an eye model of a simulation program (ZEMAX) and the modulation transfer function (MTF) on the retina was determined. A small pupil with a diameter of 3.0 mm was chosen here in order to achieve the highest possible interference component in the pattern within the pupil. In this example, the marking points 57 are completely opaque absorbers—in practice, black points for example. FIG. 8 shows the intensity distribution immediately downstream of the marked area. The pattern corresponds exactly to the example from FIGS. 7 and 7a. However, FIG. 9 shows that the MTF is practically unaffected by the central point grid and remains close to the diffraction limit.

Finally, FIG. 10 illustrates a machine reading system 200 according to the invention for recording and decoding a coded point grid made of marking points on an ophthalmological implant 100, for example on an intraocular lens, which machine reading system is part of a corresponding surgical microscope 250.

This example embodiment of a machine reading system 200 according to the invention comprises an illumination system 210 for illuminating a digital product identification 130 of an intraocular lens in the form of a machine-readable coded point grid made of marking points 135, a camera system 220 for recording structures of the machine-readable coded point grid made of marking points 135 which have been rendered detectable by use of the illumination, and an analysis unit 230 for capturing and evaluating an image, recorded by the camera system 220, of the structures of the machine-readable coded point grid made of marking points 135 rendered detectable by use of the illumination and for decoding of the digital product identification 130 of the intraocular lens, for example the type and the refractive power, for identifying the ophthalmological implant 100 from this image, and also a display and/or output apparatus 240 for displaying and/or outputting the decoded identification data of the ophthalmological implant 100.

In this case, this example embodiment of the machine reading system 200 according to the invention can also decode intraocular lenses which have already been implanted in a patient's eye 300.

FIG. 11 shows the use of a digital product identification 130, here a coded marker, for example in the form of the machine-readable coded point grid made of marking points 135, for verification of the ophthalmological implant 100 during implantation and in vivo post implantation. Already during the implantation and in vivo post implantation, there are many different occasions when a verification of the implant 100 and hence traceability is helpful. To this end, the ophthalmological implant 100 is capable of being connected to various pieces of equipment such as the surgical microscope 250 and various diagnostic equipment 260 via the digital product identification 130. These establish a contactless connection to the registry 270, which can be located on an internal server or in the cloud.

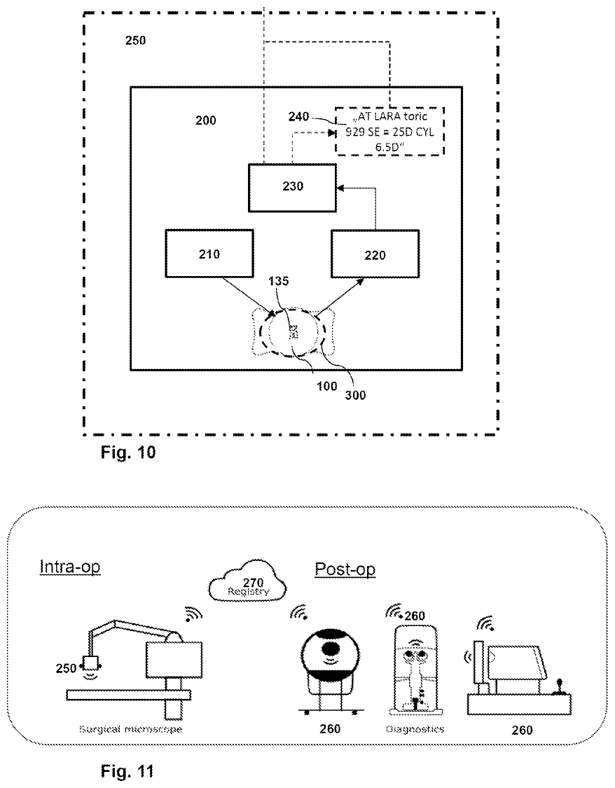

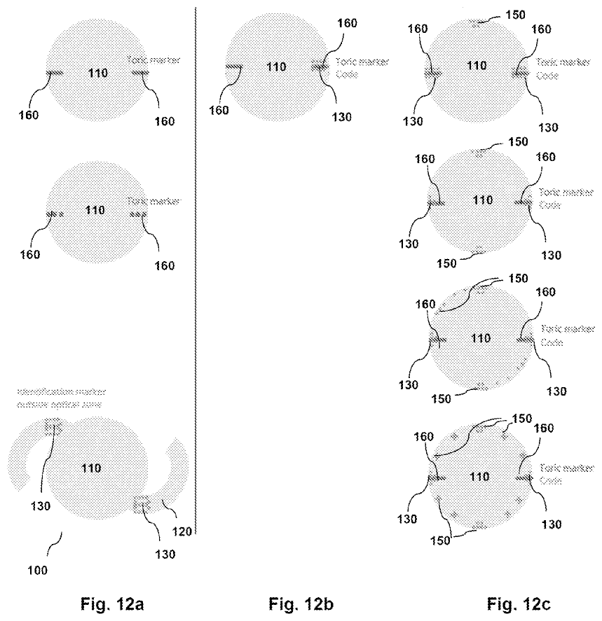

FIG. 12a illustrates a digital product identification 130 with toric marker 160 on an ophthalmological implant 100 according to the prior art: The digital product identification 130 is located in the region of the haptic 120 of the ophthalmological implant 100 close to the optically imaging element 110. Toric markers 160 are arranged at the edge of the optically imaging element 110.

FIG. 12b shows a digital product identification 130 with toric marker 160 according to an example embodiment of the ophthalmological implant 100 according to the invention, while FIG. 12c illustrates a digital product identification 130 with toric marker 160 and alignment aid 150 according to further example embodiments of the ophthalmological implant 100 according to the invention.

In general, a coded marker on an ophthalmological implant 100, for example on an IOL, can be any type of visually recognizable coded information that offers the above-described functions.

Examples of such coded information include, inter alia, (i) standard codes such as linear barcodes or matrix (2D) barcodes including point code, QR code, or (ii) advanced codes such as 3D matrix codes. The codes can differ in the number, size or width of the (individual) elements (e.g., pixels), the overall size of the code, the distances between the elements and the orientation of the elements within the code. The coded marker on an IOL is formed by a machine-readable pattern. Such a pattern can be recognized under different types of illumination, for example under normal white light illumination, fluorescent illumination or laser illumination.

The digital product identification 130 using coded markers is now attached to the IOL in such a way that it is recognizable during implantation and post operation. In the embodiments shown here, the coded marker is located at the edge of the optically imaging element 110 of an IOL, which is generally accessible by dilating the pupil. The coded marker, for example the machine-readable coded point grid made of marking points 135 contains information such as the specification data of the respective ophthalmological implant 100 (in the case of an IOL, for example diopter, type, manufacturer, model, material, toric axis). However, the specification data can also be represented by a unique identifier that enables the data to be retrieved from a database.

A coded marker as proposed in the invention not only enables a reliable identification of the IOL, but also, in example embodiments as illustrated in FIGS. 12b and 12c, the recognition of the IOL position during the operation. The use of a coded marker enables the provision of IOL design specification data (including IOL geometry) and actual IOL position data. The information supplied by the coded marker opens up new possibilities for computer-assisted optimization of IOL positioning. The coded marker represents geometric data of the individual implant 100, which data enable computer-aided recognition of the position of the said implant. On account of the coded nature of the marker, even a subset of the recognized features of a coded marker provides useful information relating to a more stable and precise alignment of an IOL in the eye. The coded marker includes features for error detection, error tolerance, and ideally error correction.

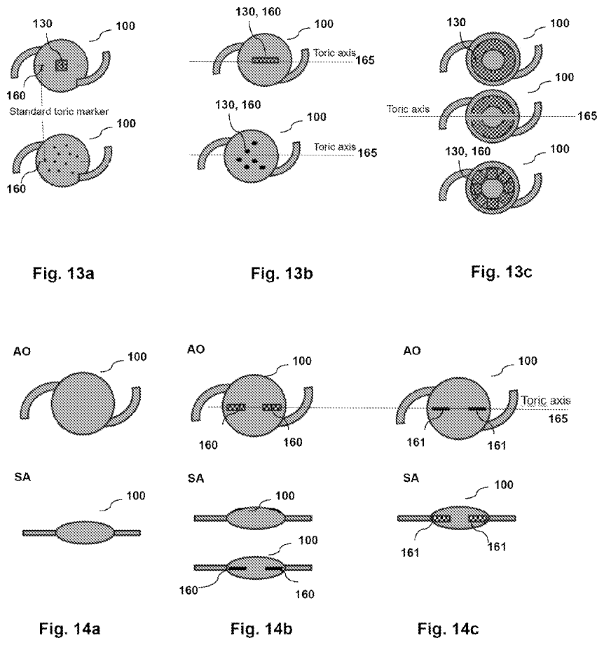

FIGS. 13a to 13c depict digital product identifications 130 with toric marker 160 in the optically effective zone of the optically imaging element 110 in further different example embodiments of the ophthalmological implant according to the invention 100. These figures describe embodiments of machine-readable coded point grids 135, that is to say data matrix codes or coded markers, and their variations for use within the optically effective zone 115 or the optical zone of 4.4 mm. This is advantageous for example to have easier access to the code in the implanted state, since dilation of the pupil is not required. In this case, it is necessary to ensure that no negative effects on the optical performance of the ophthalmological implant 100, for example an intraocular lens, are passed on to the patient. The solution here is to increase and/or randomize the spacing between the marking points and/or keep the size of the marking points small. In this case, FIG. 13a illustrates a conventional toric marker 160 from the prior art at the outermost edge of the optically imaging element 110 in combination with the digital product identification 130, described here according to the invention, in the form of a machine-readable coded point grid made of marking points 135 in the optically effective zone 115 of the optically imaging element 110 of the ophthalmological implant 100, while in FIG. 13b the toric marker 160 is integrated, in a manner according to the invention, into the digital product identification 130 in the form of a machine-readable coded point grid made of marking points 135 in the optically effective zone 115 of the optically imaging element 110 of the ophthalmological implant 100. In the lower of the two examples, in particular, the alignment is not only implemented on a macrostructure (the shape of the coded marker), but the marking points have an elliptical shape, with the long axis of the ellipse running parallel to the toric axis in this case.

To indicate the toricity of an intraocular lens or other lens characteristics (such as haptic or modular lens connection sites), the points can be extended in that direction. In addition, as shown in FIG. 13c, an optical data matrix code can be used to block the light in a desired manner, as is the case with pinhole IOLs to achieve greater depth of field. Here, the machine-readable coded point grid made of marking points 135 or, more generally, the data matrix codes form the optical mask, or a code is applied to the light-blocking mask of a pinhole IOL. This is advantageous because the optical disadvantages of a coded marker or data matrix code on a mask are dispensed with.

FIGS. 14a to 14c illustrate digital product identifications 130 with toric marker in further different example embodiments of the ophthalmological implant according to the invention—for use from the view from above AO and for use in an axial view SA.

The toric marker 160, 161 is located on the surface of the optically imaging element 110 of the ophthalmological implant 100, for example an intraocular lens, or in the volume of the optically imaging element 110 (i.e., in the material). The toric marker 160, here for example in the form of a QR code, is for example readable in the view from above, AO, as shown in FIG. 14b, allowing access via a camera in production and in logistics, and via a surgical microscope 250, a slit lamp 260 or biometric equipment 260 in the implanted state. Alternatively, the toric marker 161, optionally also the data matrix code, can be read in an axial view (i.e., a side view, SA), as shown in FIG. 14c, which is accessible in the case of tomographic measurements. In this embodiment, the code is not readable in top view (view from above, AO) but has less impact on dysphotopsia when in the implanted state.

In general, toric markers 160 for toric IOLs are well established with no reports of dysphotopsia, which has the advantage that this shape and area can be used for a coded marker in the form of a data matrix code in the implanted state of an IOL with dilated pupils. This invention can be used for all IOLs, not just toric IOLs, and allows the rotation of the ophthalmological implant 100 to be tracked.

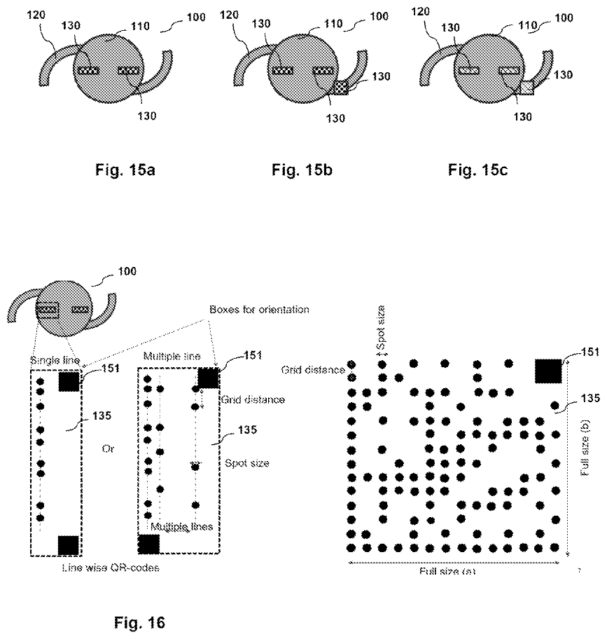

FIGS. 15a to 15c illustrate a digital product identification 130 in the form of a coded marker, for example a machine-readable coded point grid made of marking points 135, in further different example embodiments of the ophthalmological implant 100 according to the invention.

In this case, FIG. 15a shows two identical elongated coded markers in the form of QR codes on an intraocular lens 100. Since a conventional toric marker 160 contains two lines, both can be used as a UDI. Additional codes on the surface of the haptic 120 may be advantageous. All codes can have the same content, or different content to reduce the data density of a code.

FIG. 15b shows two identical elongated coded markers in the form of QR codes on an intraocular lens 100, in combination with a QR code on the haptic. FIG. 15c illustrates two non-identical elongated coded markers in the form of QR codes on an intraocular lens 100, in combination with a QR code on the haptic.

FIG. 16 shows different arrangements of the machine-readable coded point grid made of marking points 135 of a digital product identification 130.

In this case, the QR code designs are rectangular or square in UDI-compliant grid pattern, which is easier to generate than that of FIGS. 6 and 7, but retains a certain periodicity. The number of rows of data matrix codes varies between the individual examples in this case. Additional features show the alignment of the data matrix codes, improving the detection of equipment used to rotate and align the IOL during surgery. Here, additional functions of microscopes can be used to align the IOL. The codes shown here have—in addition to the fact that a basic alignment on the basis of the machine-readable coded point grid made of marking points 135 is possible in principle—additional orientation boxes 151 that support the alignment of the corresponding ophthalmological implant 100.

The tables of FIGS. 17a and 17b present a size estimation for M3 and M4 UDI codes, respectively. In order to estimate the size of a coded marker, but also for example the size of a toric marker, calculations are made for different marking point sizes, the number of columns, and the number of rows. Various combinations of row sizes, column sizes, and point sizes for the top view AO and the axial view SA are shown for an M3 code (see FIG. 17a) and an M4 code (see FIG. 17b, with M4 comprising the full content of the UDI, including SN, date of manufacture, place of manufacture and expiration date, and a checksum length). There are combinations of rows, columns, and point sizes that meet the requirements of the ISO standard regarding the arrangement of a corresponding marker on the optically imaging element 110, for example in the edge region thereof. However, a small spot size of 20 μm/25 μm to 50 μm is also advantageous in terms of resolution and contrast. The example combinations are highlighted in FIGS. 17a and 17b. An axial resolution of ˜5 μm is possible for conventional OCT. This would be a lower limit for point sizes.

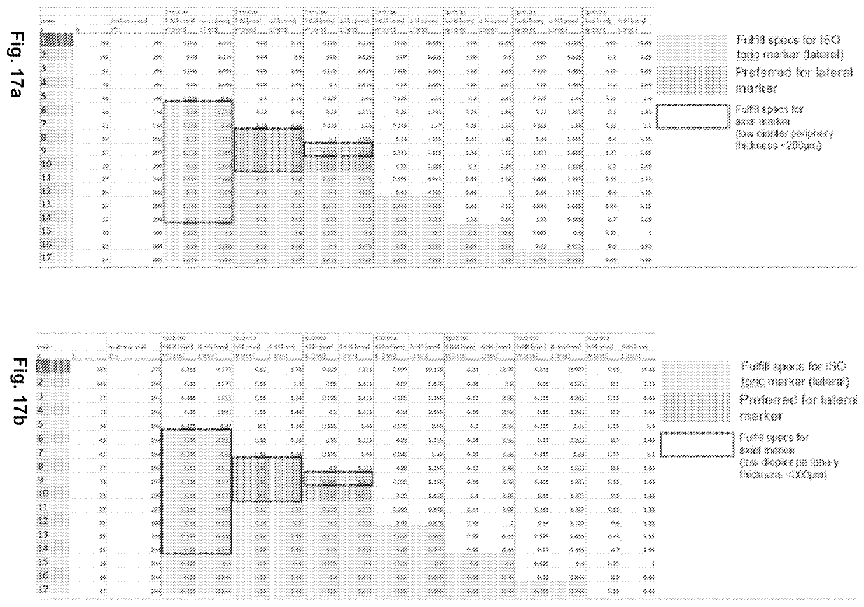

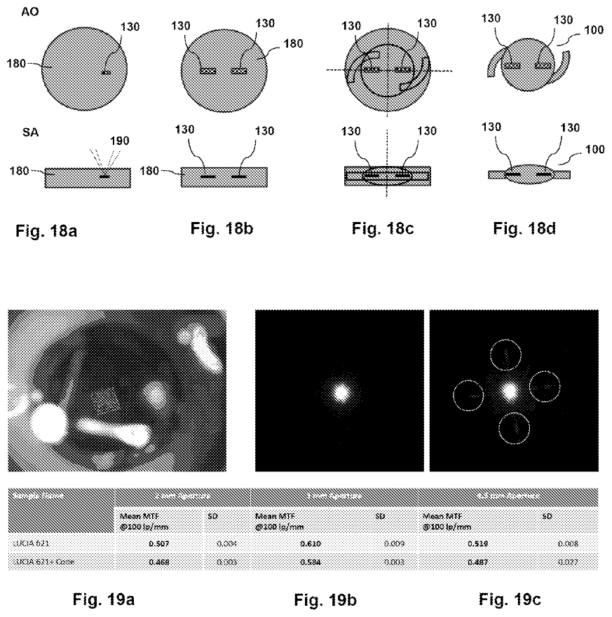

For the traceability of an ophthalmological implant 100 until production, in particular of the optically imaging element 110, within the scope of manufacture, coded markers, for example a machine-readable coded point grid made of marking points 135, can be applied in or on the material as a digital product identification 130 by laser engraving using a laser 190. FIGS. 18a to 18d describe an example variant of a manufacturing method of an intraocular lens with a coded marker as a digital product identification 130, but also as an alignment aid or as a toric marker, which is arranged in the volume of the optically imaging element 110. Here, the codes are first written into the material blank 180 (also called a blank); see FIGS. 18a and 18b. Since the code or codes are in the interior of the material, the ophthalmological implant 100 can already be tracked throughout the entire manufacturing process, including turning, milling, sterilizing, and packaging; see FIGS. 18c and 18d. In the material, the code is protected from any abrasion during diamond turning and diamond milling. In the example shown here, the code of the digital product identification 130 is where the toric markers 160 will be located in the finished product.

Another advantage is that a data matrix code in the interior of the material improves readout during production, logistics, and in the implanted state. For example, toric markers 160 on the surface of the optically imaging element 110, for example on a lens surface, at high diopters, and on the cornea, may appear deformed due to optical projection, making alignment during implantation of the ophthalmological implant 100 more difficult. Moreover, the toric markers 160 can also be used to align the material blank 180 during the production of the ophthalmological implant 100 in order to ensure toricity and haptic are on the correct axis.

It should be mentioned that the processing with regard to tilting and centering must be very precise, especially for the precisely timed alignment of a non-spherical intraocular lens. In the event of the lens not being perfectly aligned, this information must be linked to the serial number and stored both online (accessed via the database) and offline (box label).

Alternatively, the marker can also be attached directly after the processing or after the polishing of the ophthalmological implant 100. In this context, the marker can still be embedded into the material or applied to the surface.

In addition to conventional laser engraving, alternative techniques can also be used to produce the data matrix codes shown. One possibility would be the photochemical generation of metal (e.g., silver, gold) nanoparticles or silicon nanoparticles within the biomaterial matrix by way of two-photon absorption.

FIGS. 19a to 19c depict recordings of a Zeiss Lucia 621 intraocular lens implanted in an ISO eye and having a coded marker on the surface of the optically imaging element 110 (FIG. 19a) and the halo/glare test, the latter both without this coded marker (FIG. 19b) and with this coded marker (FIG. 19c). This relates to a laser engraved QR code (M4) on the IOL surface in an ISO eye used for bench testing. The code was introduced, tested, and analyzed as a 50 μm standard unique device identifier (UDI) pattern directly in the center of the optically effective zone 115.

The code is clearly visible through a microscope. Modulation transfer function (MTF) values for 100 lp/mm are close to those of an IOL that has not been provided with a coded marker. Thus, only minor problems in terms of halo, glare and visual impairment can be determined in the case of this conventional laser-engraved code. Small lattice effects (small dysphotopsias) are visible due to the periodicity of the code. However, the test limits for such an ophthalmological implant 100 are met. These effects could be further minimized by positioning at the periphery of the optically imaging element 110 or further randomization of the pattern during the creation of the digital product identification 130.

Since the marking points are located in a Fourier plane, they are not projected onto the retina in focus. Here only visual acuity is reduced by the nature of the “blocking” of light rays in the central optical zone, which leads to a loss of contrast. About 0.5% of the light is blocked in an estimate for an M4 code (17×17) with a square marking point size of 25 μm and a central optical zone of 4.4 mm; this is negligible and has no impact on the MTF function. In order to reduce diffraction effects due to the periodicity of the point pattern of the code, the lattice spacing must be greater than the size of the marking points (sometime also called spot size). This is the case in the present example.

The aforementioned features of the invention, which are explained in various example embodiments, can be used not only in the combinations specified in an example manner but also in other combinations or on their own, without departing from the scope of the present invention.

A description of a piece of equipment relating to method features is analogously applicable to the corresponding method with respect to these features, while method features correspondingly represent functional features of the equipment described.

### REFERENCE SIGNS

0 to 36: Base lattice point numbers
1.1, 1.2, 1.3, 1.4: Possible positions of the marking point of the first base lattice point
51 Sector/cell
52 Radial zone
53 Radial direction
54 Sectoral direction
55 Crossing point/possible position for marking point
56 Base lattice point
57 Marking point
58 Reference mark
100 Ophthalmological implant
110 Optically imaging element
115 Optically effective zone
120 Haptic
130 Digital product identification
135 Machine-readable coded point grid made of marking points
140 Virtual polar or Cartesian base lattice
150 Alignment aid
151 Orientation box
160 Toric marker for view from above
161 Toric marker for view from the side/axial view
165 Toric axis
180 Blank/Material blank
190 Laser
200 Machine reading system
210 Illumination system
220 Camera system
230 Analysis unit
240 Output apparatus
250 Surgical microscope
260 Diagnostic equipment
270 Registry
300 Patient's eye
AO View from above/lateral view
SA Side view/axial view

1.-23. (canceled)

24. An ophthalmological implant or an intraocular lens, comprising:
an optically imaging element, including a central optical lens having an optically effective zone, and comprising a haptic adjoining the optically imaging element;
and with a digital product identification of the ophthalmological implant, the digital product identification including a type, a refractive power, a database key or a combination thereof, being arranged on the optically imaging element, within the optically effective zone;
wherein the digital product identification presents a coded point grid made of marking points that is machine-readable in the visible light range and has a pseudo-random, irregular character.

25. The ophthalmological implant as claimed in claim 24, wherein the machine-readable coded point grid made of marking points is arranged centrally within the optically effective zone of the optically imaging element.

26. The ophthalmological implant as claimed in claim 24, wherein the coded point grid is constructed from marking points such that
a virtual polar or Cartesian base lattice is arranged on the optically imaging element, within the optically effective zone of the optically imaging element, that describes similar sectors or similar cells, each sector or cell having a defined base lattice point of the sector or the cell, as a result, and a real marking point of the coded point grid is arranged in each sector or each cell at a position which has an offset to the base lattice point, the offset in each sector or each cell being in one of four possible directions, which run pairwise opposite to each other, and having a defined distance to the base lattice point.

27. The ophthalmological implant as claimed in claim 26, wherein the sector or the cell with the base lattice point provides four states wherein a respective location of the marking point in one of four positions around the base lattice point.

28. The ophthalmological implant as claimed in claim 27, wherein the sector or the cell with the base lattice point provides a fifth state defined by the absence of a marking point at one of the four possible positions around the base lattice point.

29. The ophthalmological implant as claimed in claim 27, wherein further states are defined in the sector or the cell with the base lattice point by further offset directions, further defined distances of the offset of the marking point to the base lattice point or both.

30. The ophthalmological implant as claimed in claim 24, wherein a proportion of an area of the marking points to the total area of the optically imaging element is selected from a group consisting of less than 2%, less than 1%, and less than 0.5%, and wherein the proportion of the area of the marking points to the area of the optically effective zone of the optically imaging element is selected from a group consisting of less than 8%, less than 4%, and less than 2% or a combination of the foregoing.

31. The ophthalmological implant as claimed in claim 24, wherein the machine-readable coded point grid has structural marking points.

32. The ophthalmological implant as claimed in claim 24, further comprising a supplemented product identification which, in addition to the original product identification, has information for checksums and error correction methods.

33. The ophthalmological implant as claimed in claim 24, further comprising one or more reference marks at a defined distance from the machine-readable coded point grid made of marking points.

34. The ophthalmological implant as claimed in claim 24, wherein the machine-readable coded point grid made of marking points is arranged on the optically imaging element, in the optically imaging element or both.

35. The ophthalmological implant as claimed in claim 24, further comprising an alignment aid, the alignment aid being arranged within the optically effective zone and comprising or consisting of the machine-readable coded point grid made of marking points.

36. The ophthalmological implant as claimed in claim 24, further comprising a toric marker which is readable in a view from above, a toric marker which is readable in an axial view or both.

37. A machine reading system for capturing and decoding the digital product identification in the form of a coded point grid made of marking points on an ophthalmological implant as claimed in claim 1, comprising
a camera system that records structures of the machine-readable coded point grid made of marking points on the ophthalmological implant, and
an analyser that captures and evaluates a camera system recorded image of the structures of the machine-readable coded point grid made of marking points, and that decodes the digital product identification of the ophthalmological implant.

38. The machine reading system as claimed in claim 37, further comprising a connected database system, wherein the digital product identification contains a database key and the database key is assigned the product information of the ophthalmological implant in the database system.

39. The machine reading system as claimed in claim 37, which is part of a surgical microscope or a slit lamp.

40. A method to produce an ophthalmological implant with a digital identifier as claimed in claim 24, comprising, generating the machine-readable coded point grid made of marking points for digital product identification purposes on the ophthalmological implant, during or after the production of the ophthalmological implant.

41. The method as claimed in claim 40, wherein the production of the ophthalmological implant further comprises:
either labelling the ophthalmological implant with the machine-readable coded point grid made of marking points in an early phase of production before the ophthalmological implant is complete,
or labelling the ophthalmological implant with the machine-readable coded point grid made of marking points directly after the production has been completed, but still within the same step with a similar tool used to produce the ophthalmological implant.

42. The method as claimed in claim 41, further comprising introducing the machine-readable coded point grid made of marking points into the surface of the ophthalmological implant using a CNC-controlled drilling or milling tool during or after the production of the ophthalmological implant, and selecting the drilling or milling tool to have a tool diameter of less than 0.4 mm.

43. The method as claimed in claim 40, further comprising applying the machine-readable coded point grid made of marking points either by application of laser processing by ablation or disruption or by application of printing methods, using biocompatible chromophores or pigments.

44. The method as claimed in claim 40, further comprising converting a product identification or a supplemented product identification into grid coordinates for the physical product identification using a machine-readable coded point grid made of marking points.

45. The method as claimed in claim 40, further comprising, during or after the generation of the machine-readable coded point grid made of marking points for digital product identification, storing the machine-readable coded point grid in a manufacturer database which is linkable to an electronic patient file and/or another data collection point for medical or official purposes.

46. The method as claimed in claim 40, further comprising preceding the generation of the machine-readable coded point grid made of marking points for digital product identification by storing the product information of the ophthalmological implant in a database system and generating a database key relating to this product information and contained in the digital product identification.
